(19) United States
(12) Patent Application Publication
(10) Pub. No.: US 2011/0264029 A1
(43) Pub. Date: Oct. 27, 2011

(54) IONTOPHORETIC DELIVERY SYSTEM

(75) Inventor: Alex Savelij Rivlin, Oakville (CA)

(73) Assignee: Stanley E. Kopman, Toronto (CA)

(21) Appl. No.: 12/768,006

(22) Filed: Apr. 27, 2010

(51) Int. Cl. A61N 1/30

(52) U.S. Cl. 604/21; 604/501; 604/20

(57) ABSTRACT

Methods, systems and other embodiments associated with administering ionotophoretic medicines are presented. A method of administering ionotophoretic medicines includes applying a voltage across a body tissue and an iontophoretic solution including medicine. At least some of the iontophoretic solution including medicine is then injected into the body tissue without requiring the iontophoretic transportation of the iontophoretic solution including medicine through the skin. The iontophoretic solution is typically injected while the voltage is applied. The voltage causes an ionic current of iontophoretic medicine to flow which disperses the medicine within the body tissue toward body tissue connected to the voltage.

### BACKGROUND OF THE INVENTION

1. Technical Field

The present invention relates generally to systems and methods for transdermally delivering a medical solution to a patient. More particularly, the systems and methods relate to using an ionic current for delivering the medical solution to the patient. Specifically, the methods and systems of the present invention involve an iontophoretic system and method of delivering medicine.

2. Background Information

An iontophoretic process of using a directed electrical current to deliver a medical solution was introduced by Giovanni Giuseppe Veratti in 1747. This process was improved upon by LeDuc in 1908. In general, as shown in FIGS. 1 and 2 an iontophoretic delivery system or method creates a small electrical current between two electrodes 7, 8 (an anode and a cathode) positioned on the skin 5 of a medical patent. The current is created by connecting each of the electrode to one terminal of a power supply 10. A medical solution containing either positive or negative ions can be placed on the skin under one of the electrodes 7, 8. The electrical current between the electrodes will cause the medical solution to be transported into the skin, through the interior tissue 3 of the patient and toward the other electrode. Iontophoretic delivery systems can be used for transdermal delivery of steroids to joints, for the administration of local anesthetics, for the transdermal transport of insulin and the like.

With iontophoretic devices, the application time and level of current flow between the anode and cathode is directly correlated to the amount of medicine delivered. The efficiency of medicine delivery in an iontophoretic system can be measured by the proportion of current carried by medicine molecules, relative to the current carried by competing non-medication ions having the same charge as the medication.

Iontophoretic delivery devices conventionally have included two electrodes 7,8 attached to a patient, each connected via a wire to a microprocessor controlled electrical instrument. Medication is placed under one or both of the electrodes, for delivery into the body as the instrument is activated. The instrument is designed to regulate current flow and application time. Power for these devices is usually provided by DC batteries, which when providing power for the microprocessor controlled circuitry allow application of a voltage to the electrodes to create a regulated current flow. A typical application period for creation of skin anesthesia is approximately 10-20 minutes, which consumes instrument, caregiver, and patient time.

More recently, wearable iontophoretic transport systems have been developed in which the electrical circuitry and power supplies are integrated into a single patch. These systems are advantageous in that they do not have external wires, and they are much smaller in size. Typically, medicine ions are delivered into the body from an aqueous “medicine” reservoir contained in the iontophoretic device, and counter ions of opposite charge are delivered from a “counter” reservoir. An important step in iontophoresis involves the process for incorporation of medicine ions and counter ions into the device. In these types of systems it is well known that if such a device is improperly loaded, the device will not perform as desired.

Often, medicine/ion solutions are stored remotely in bulk quantity and introduced to an absorbent layer of the iontophoresis electrode at the time of use. An advantage to this approach is that the electrodes are packaged and stored in a dry state, which is optimal for shelf life. A disadvantage to this approach is that the electrodes can be easily over-filled or under-filled. Therefore, the uses of these devices may require highly trained personnel to be sure a proper amount of medical solution is actually administered. Additionally, because the medicine solution is stored separately from the electrodes, management of two inventories is required.

To avoid the need for users to incorporate the aqueous medicine or ion reservoir at the time of use, the medicine solution can be pre-packaged into the electrode. Unfortunately, this inevitably reduces shelf life. During storage, moisture emanating from the medicine solution can be absorbed into adjacent materials, resulting in corrosion of metallic components, degradation of power sources, and inadequate hydration of the medicine pad. For example, some iontopohoric delivery systems include a device where an aqueous reservoir is stored in contact with an electrode assembly. In these systems, a dry medicament layer is introduced to the aqueous reservoir at the time of use. Unfortunately, in these systems the electrode is still stored in a wet environment, and is therefore susceptible to corrosive deterioration.

In other iontophoretic delivery systems, medicine solutions are co-packaged with the iontophoretic device and positioned apart from the electrodes and other metallic components until an “activation” step is implemented at the time of use. In these systems, a co-packaged electrolyte constituent liquid is stored remotely from the electrode, in a rupturable container and a mechanical action step at the time of use induces a fluid transfer to a receiving reservoir adjacent to the electrodes. These devices are mechanically complex, and can fail if, for example, the package is squeezed during shipping, the container breaks and fluids are pre-maturely released. Other failure modes include compromising the fluid delivery path during storage. For example, if outgassing hydrophobic plasticizer material is absorbed into the fluid channel, this will inhibiting the transfer of fluid at the time of use. Therefore, a better way of iontophoretic transport of medicine is desired.

### BRIEF DESCRIPTION OF THE DRAWINGS

One or more preferred embodiments that illustrate the best mode(s) are set forth in the drawings and in the following description. The appended claims particularly and distinctly point out and set forth the invention.

The accompanying drawings, which are incorporated in and constitute a part of the specification, illustrate various example methods, and other example embodiments of various aspects of the invention. It will be appreciated that the illustrated element boundaries (e.g., boxes, groups of boxes, or other shapes) in the figures represent one example of the boundaries. One of ordinary skill in the art will appreciate that in some examples one element may be designed as multiple elements or that multiple elements may be designed as one element. In some examples, an element shown as an internal component of another element may be implemented as an external component and vice versa. Furthermore, elements may not be drawn to scale.

FIG. 1 illustrates a prior art system for administering an iontophoretic solution containing a medicine.

FIG. 2 illustrates another configuration of the prior art system for administering the iontophoretic solution containing the medicine.

FIG. 3 illustrates the preferred embodiment of a system for administering an iontophoretic solution containing a medicine.

FIG. 4 illustrates a second embodiment that is a method of administering an iontophoretic solution containing a medicine.

FIG. 5 illustrates a third embodiment that is another method of administering the iontophoretic solution containing the medicine.

Similar numbers refer to similar parts throughout the drawings.

### DETAILED DESCRIPTION

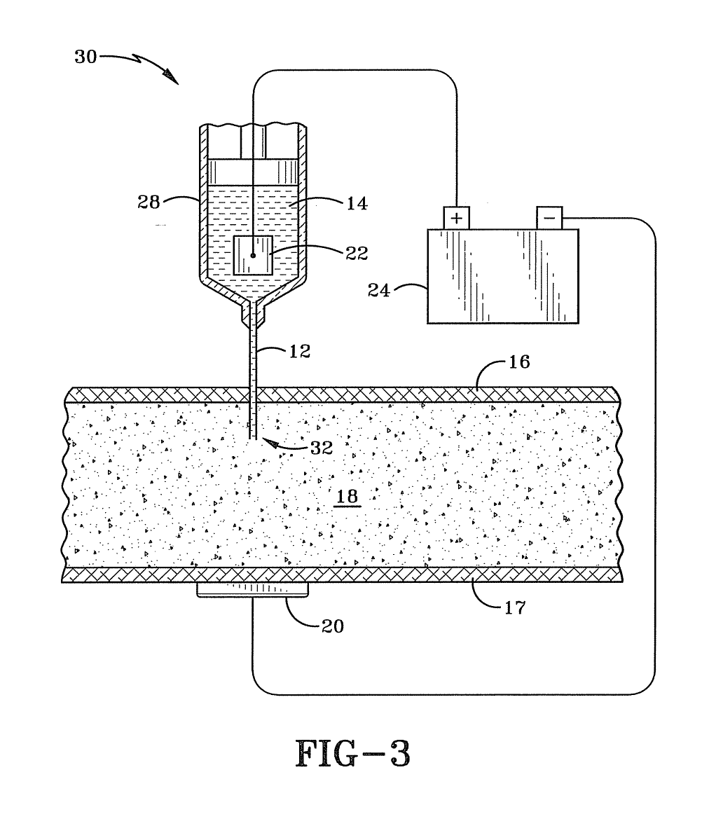

FIG. 3 illustrates a first embodiment that is the preferred embodiment of a system 30 for iontophoretically administering an iontophoretic medicine. The system 30 improves over the prior art systems discussed earlier by at least partially immersing a submersible electrode 22 into an ionized (e.g. iontophoretic) solution 14 with a medicine and injecting the medicine into body tissue 18 beneath the skin 16. This allows for precise initial placement of the medicine within the body as well as providing a way to control the dosage amount of the medicine entering the body. An ionic current is created between the submersible electrode 22 and another external electrode 20 placed on the surface of the skin 17 on the body. This current will cause the iontophoretic medicine to flow from the injection point 32 toward the external electrode 20. The concentration of the medicine may be increased or decreased by controlling the ionic current with the voltage applied by a power supply 24. The voltage can also be removed when the ionic solution 14 reaches a target location in the body tissue 18. In general, the power supply 24 will apply voltage to produce an ionic current in the range of about 0.05 milli-amps (mAs), but other values can be used.

This system 30 allows for appropriate drugs to be transported at high concentration to the site of interest when the speed of drug application and/or concentration of the drug are important. The system 30 maximizes the concentration of the drug at the site of interest while minimizing the systemic concentration of medicine, thereby reducing side effects of the medicine.

A chamber 28 containing the iontophoretic solution may be formed in a syringe and the syringe may be connected to a needle 12. The iontophoretic solution with the medicine can be injected into the body of biological tissue 18 through the needle 12. For example, the system 30 can be used to inject the iontophoretic solution 14 with medicine, at least one millimeter (1 mm) under the skin 16. Those of ordinary skill in the art will appreciate that the system can inject the iontophoretic solution 14 to other different depths under the skin 16.

The system 30 can also inect an ionized solution 14 that has tracer material in the solution in addition to the medicine. The tracer material can then be monitored to determine when the ionic solution has reached a target location or when the ionic solution 14 and medicine have filled a specific region of the body. The voltage can be applied at the terminals 20, 22 until the ionic solution 14 has reached the target location or has filled the specific region.

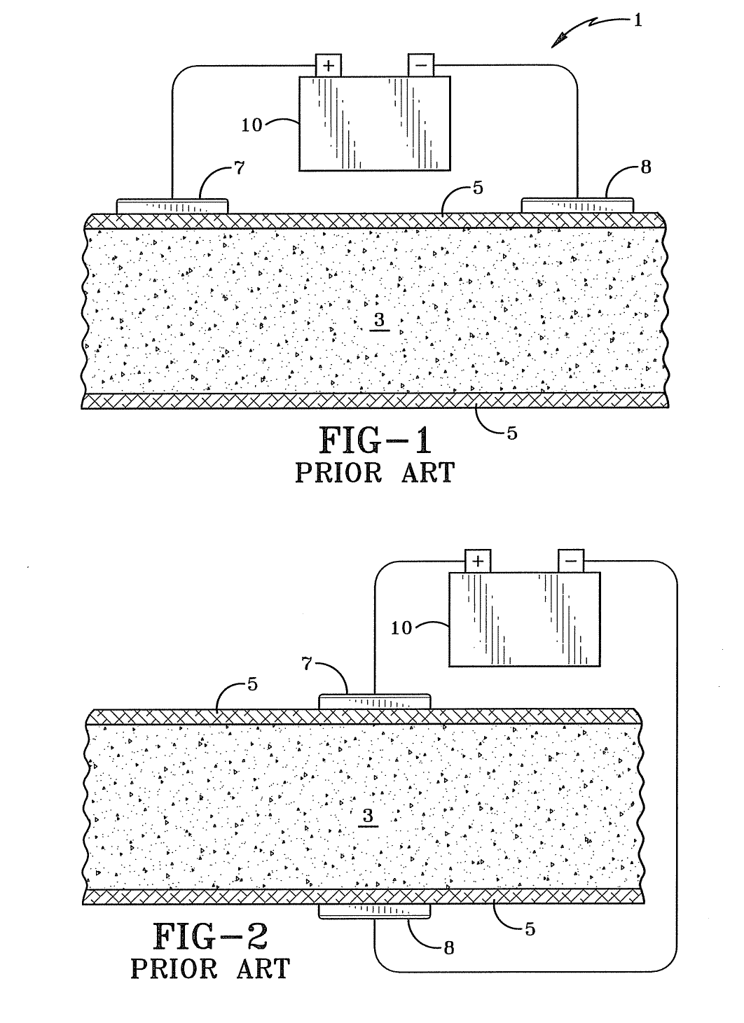

FIG. 3 illustrates one example placement of the preferred embodiment with the submersible electrode 22 and chamber 28 injecting the iontophoretic solution 14 on one side of the tissue 18 and the external terminal 20 on another opposite side of the tissue 18. This arrangement transports the iontophoretic solution 14 ionically from one side of the tissue 18 toward the other side. However, similar to FIG. 1, the iontophoretic solution 14 can be injected on one side of the tissue and the external terminal 20 can be placed on the same side of the tissue. This transports the iontophoretic solution 14 from the needle 12 through tissue material on this adjacent side of the tissue toward the external terminal 20. The arrangement of the external electrode 20 with respect to the internal electrode 22 and injection point 32 can be a variety of other configurations as understood by one of ordinary skill in the art.

Example methods may be better appreciated with reference to flow diagrams. While for purposes of simplicity of explanation, the illustrated methodologies are shown and described as a series of blocks, it is to be appreciated that the methodologies are not limited by the order of the blocks, as some blocks can occur in different orders and/or concurrently with other blocks from that shown and described. Moreover, less than all the illustrated blocks may be required to implement an example methodology. Blocks may be combined or separated into multiple components. Furthermore, additional and/or alternative methodologies can employ additional, not illustrated blocks.

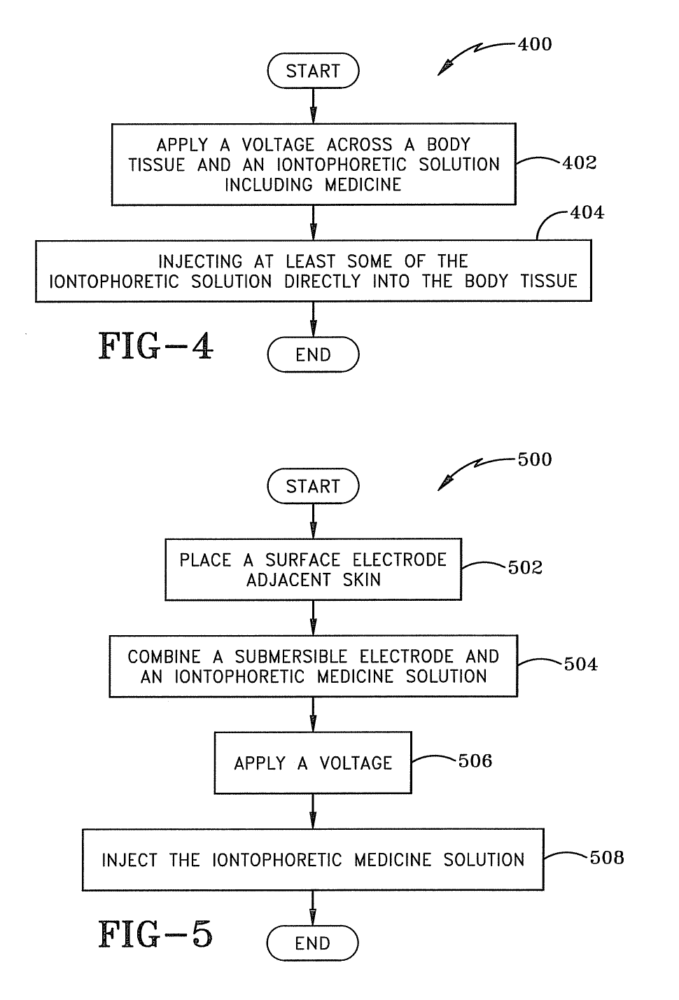

FIG. 4 illustrates a second embodiment that is a method 400 of administering an iontophoretic solution with a medicine. The method 400 applies a voltage across a body tissue and an iontophoretic solution including medicine, at 402. This voltage will cause the iontophoretic transportation of the iontophoretic solution with the medicine once the solution is injected into the body. At least some of the iontophoretic solution is injected, at 404, directly into the body tissue without requiring the iontophoretic transportation of the iontophoretic solution including medicine through the skin as in prior art systems that do not inject the iontophoretic solution into the skin. Typically, the injecting, at 404, is performed while the voltage is applied.

As mentioned earlier, the iontophoretic solution can be directly injected into the body tissue with a needle of a syringe. When the iontophoretic solution and medicine is directly injected into tissue below the keratin layer of the skin, there is no need for the iontophoretic solution to be iontophoretically transported through the keratin layer of the skin. More accurate amounts of medicine can be administered to tissue areas when the medicine does not initially have to be iontophorically transported through skin. Unlike other iontophoretic delivery methods, the amount of injected solution in this method can be quantified and the time of the delivery and can be controlled by the ionic current.

Additionally, the pain of injection can be reduced as compared to traditional methods of injecting medicine into tissue without using iontophoretic approaches. This is because in traditional injection by syringe a thin needle is first inserted and then during the injection phase the medicine is delivered. Most pain is the result of the injection phase when the medicine is pushed into the tissue and surrounding tissue structures are compressed which often breaches sensory nerves. Method 400 greatly eliminates this pain because as the medicine is injected, an ionic current disperses the iontophoretic solution and medicine.

As previously mentioned when discussing the system 30 of FIG. 3, a tracer material can be added to the iontophoretic solution. As understood by those of ordinary skill in the art, a tracer is a substance, such as a dye, a radioactive isotope or another compound that mixed into the iontophoretic solution and is injected into the tissue as part of the iontophoretic solution. The tracer is then followed through a biological or chemical process, by virtue of its color, radioactive signature, or other distinguishing physical property. Thus, the method 400 can track the tracer material while the iontophoretic solution including medicine travels in the body tissue responsive to the voltage. The medicine can be tracked in this way and the method 400 can then control how deep the medicine penetrates the body tissue by controlling the voltage.

FIG. 5 illustrates another method 500 associated with injecting ionic solutions of medicine that is a variation of method 400 in FIG. 4. The method 500 begins by placing a surface electrode adjacent skin covering biological tissue, at 502. A submersible electrode and an iontophoretic medicine solution are combined, at 504. The submersible electrode and the medicine solution are combined in a way so that the submersible electrode is submersed in the medicine solution so that the electrode is at least partially in contact with the solution. Note that the submersible electrode does not have to be totally submersed and can be partially submersed. This electrode can also at least partially in contact with the ontophoretic solution. The contact does not have to be direct physical contact. For example, the submersible electrode can be wrapped in a thin plastic film and be placed in the solution and still create an iontophoretic current as discussed below. The submersible electrode can be placed/located inside a syringe or be part of a syringe. The surface electrode and the submersible electrode form an electrical anode/cathode pair.

A voltage is applied across the surface electrode and the submersible electrode, at 506. The voltage creates an ionic current that causes the iontophoretic medicine solution to flow from its injection point toward the external electrode. The iontophoretic medicine solution is injected, at 508, into the biological tissue. The medicine solution can be injected into the biological tissue through a needle that may be part of a syringe. The injecting of the medicine solution, at 508, generally occurs while the voltage is applied.

As previously mentioned, injecting an iontophoretic medicine below the skin and creating an iontophoretic current allows the dosage and placement of the medicine to be more precisely controlled. The voltage of the ionic current of the iontophoretic medicine can be used to control the depth of the penetration of the medicine into the body after the medicine is injected below the skin.

In the foregoing description, certain terms have been used for brevity, clearness, and understanding. No unnecessary limitations are to be implied therefrom beyond the requirement of the prior art because such terms are used for descriptive purposes and are intended to be broadly construed. Therefore, the invention is not limited to the specific details, the representative embodiments, and illustrative examples shown and described. Thus, this application is intended to embrace alterations, modifications, and variations that fall within the scope of the appended claims.

Moreover, the description and illustration of the invention is an example and the invention is not limited to the exact details shown or described. References to “the preferred embodiment”, “an embodiment”, “one example”, “an example”, and so on, indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in the preferred embodiment” does not necessarily refer to the same embodiment, though it may.

What is claimed is:

1. A method comprising:
applying a voltage across a body tissue and an iontophoretic solution including medicine; and
injecting at least some of the iontophoretic solution directly into the body tissue without requiring the iontophoretic transportation of the iontophoretic solution including medicine through the skin.

2. The method of claim 1 further comprising:
controlling how deep the iontophoretic solution including medicine penetrates the body tissue by controlling the voltage.

3. The method of claim 1 wherein iontophoretic solution includes a tracer material and further comprising:
tracking the tracer material in the body tissue.

4. The method of claim 1 wherein the iontophoretic medicine contains a tracer and further comprising:
determining when the iontophoretic solution including medicine reaches a location in the body tissue based, at least in part, on the tracer; and
ceasing application of the voltage when the iontophoretic solution including medicine reaches the location.

5. The method of claim 4 further comprising:
stopping injection of the iontophoretic solution including medicine into the body tissue when the iontophoretic solution including medicine reaches the location.

6. The method of claim 1 wherein the injecting further comprises:
injecting the iontophoretic solution including medicine into the body tissue below a keratin layer of the skin.

7. The method of claim 1 wherein the injecting further comprises:
injecting the iontophoretic solution including medicine through a needle while the voltage is applied.

8. A method comprising:
placing a surface electrode adjacent skin covering biological tissue;
combining a submersible electrode and an iontophoretic medicine solution, wherein the submersible electrode is in contact with the iontophoretic medicine solution;
applying a voltage across the surface electrode and the submersible electrode; and
injecting the iontophoretic medicine solution into the biological tissue.

9. The method of claim 8 further comprising:
controlling how deep the iontophoretic medicine solution penetrates into the biological tissue based, at least in part, on the voltage.

10. The method of claim 8 wherein the submersible electrode is located inside a syringe.

11. The method of claim 8 wherein injecting further comprises:
injecting the iontophoretic medicine solution into the biological tissue through a needle.

12. The method of claim 8 wherein injecting further comprises:
injecting the iontophoretic medicine solution while the voltage is applied.

13. The method of claim 8 wherein the combining the submersible electrode and the iontophoretic medicine solution are combined in a syringe.

14. A system for iontophoretically administering a medicine comprising:
a chamber adapted to hold an iontophoretic solution, with the medicine, wherein the chamber is configured to inject at least some of the medicine beneath a skin of a biological tissue;
an external electrode configured to be placed on the skin surface of the biological tissue; and
a chamber electrode configured to be placed in the chamber and configured to be at least partially in electrical communication with the iontophoretic solution.

15. The system of claim 14 further comprising:
a needle attachable to the chamber, wherein the system is adapted to inject the iontophoretic solution with the medicine into the biological tissue through the needle.

16. The system of claim 14 wherein the external electrode and the submersible electrode are adapted to be connected between a voltage pair, and wherein an ionic flow of the medicine is created when the external electrode and the submersible electrode are connected to the pair voltage pair.

17. The system of claim 16 wherein the chamber is loaded with the iontophoretic solution and sealed to form a sealed chamber.

18. The system of claim 16 further comprising:
a first wire connecting the external electrode to a power supply;
a second wire connecting the submersible electrode to the power supply; and
wherein the system is configured to cause ionic flow of the medicine toward the external electrode when the voltage pair is applied across the external electrode and the submersible electrode, and wherein the system is configured to inject the iontophoretic solution with the medicine into the biological tissue below a keratin layer of skin.

19. The system of claim 14 further comprising:
a needle, wherein the needle is adapted to inject the iontophoretic solution with the medicine at least one milli-meter (1 mm) under the skin.

20. The system of claim 14 further comprising:
a syringe, wherein the chamber is within the syringe.
